(19) United States
(12) Patent Application Publication
(10) Pub. No.: US 2023/0326543 A1
(43) Pub. Date: Oct. 12, 2023

(54) SYSTEM, METHOD, AND COMPUTER READABLE STORAGE MEDIUM FOR AUTO-REGRESSIVE WAVENET VARIATIONAL AUTOENCODERS FOR ALIGNMENT-FREE GENERATIVE PROTEIN DESIGN AND FITNESS PREDICTION

(71) Applicant: University of Chicago, Chicago, IL (US)

(72) Inventors: Andrew L. FERGUSON, Chicago, IL (US); Niksa PRALJAK, Parma, OH (US)

(73) Assignee: University of Chicago, Chicago, IL (US)

(21) Appl. No.: 18/176,375

(22) Filed: Feb. 28, 2023

Related U.S. Application Data
(60) Provisional application No. 63/314,898, filed on Feb. 28, 2022.
(60) Provisional application No. 63/390,663, filed on Jul. 20, 2022.

(51) Int. Cl. G16B 15/20; G16B 40/20; G06N 3/0455; G06N 3/0464; G06N 3/08

(52) U.S. Cl. CPC G16B 15/20; G16B 40/20; G06N 3/0455; G06N 3/0464; G06N 3/08

(57) ABSTRACT

A system, computer readable storage medium and method for generating protein sequences, includes an encoder configured to encode a plurality of input protein sequences onto a latent space distribution, and an autoregressive generator configured to decode the latent space distribution to generate new protein sequences different from the input protein sequences. The system is trained with a loss function that includes reconstruction loss and aa mutual information maximization term.

### CROSS-REFERENCE TO RELATED APPLICATIONS

This application claims the benefit of priority to Provisional Application Nos. 63/314,898, filed Feb. 28, 2022; and 63/390,663, filed Jul. 20, 2022, the entire contents of which are incorporated herein by reference.

### STATEMENT OF ACKNOWLEDGEMENT

This invention was made with government support under EB009412 awarded by the National Institutes of Health, and 1746045 awarded by the National Science Foundation. The government has certain rights in the invention.

The inventors gratefully acknowledge support from the Machine Learning in the Chemical Sciences and Engineering program of The Camille and Henry Dreyfus Foundation.

### STATEMENT REGARDING PRIOR DISCLOSURE BY THE INVENTORS

Aspects of this technology are described in an article entitled “Auto-regressive WaveNet Variational Autoencoders for Alignment-free Generative Protein Design and Fitness Prediction,” ICLR2022 Machine Learning for Drug Discovery (2022). The article was published online Apr. 29, 2022, and is incorporated herein by reference in its entirety.

### FIELD

The present disclosure is directed to a system, method, and computer-readable storage medium for generation of novel synthetic protein sequences and for function prediction through reconstruction of input protein sequences. In particular, the disclosure is directed to a deep generative model having a dilated convolutional neural network encoder and a decoder implemented as a decoder employing dilated causal convolutions.

### BACKGROUND

Protein sequences from non-homologous families or within homologous families with high variability and diverse lengths present challenges in the construction of multiple-sequence alignments. See Ethan C Alley, Grigory Khimulya, Surojit Biswas, Mohammed AlQuraishi, and George M Church, “Unified rational protein engineering with sequence-based deep representation learning,” Nature Methods, 16(12):1315-1322, 2019; Surojit Biswas, Grigory Khimulya, Ethan C Alley, Kevin M Esvelt, and George M Church, “Low-n protein engineering with data-efficient deep learning,” Nature Methods, 18(4):389-396, 2021; and Jung-Eun Shin, Adam J Riesselman, Aaron W Kollasch, Conor McMahon, Elana Simon, Chris Sander, Aashish Manglik, Andrew C Kruse, and Debora S Marks, “Protein design and variant prediction using autoregressive generative models,” Nature Communications, 12(1):1-11, 2021, each incorporated herein by reference in their entirety.

Recently deep generative models (DGMs) have been highly successful in novel protein design and could enable an unprecedented level of control in therapeutic and industrial applications. One DGM approach is variational autoencoders (VAEs), which can infer higher-order amino acid dependencies for useful prediction of fitness effects of mutation. Additionally, such a model infers a latent space distribution, which enables the model to learn biologically meaningful representations. Another example of a DGM approach is autoregressive models, commonly implemented in language or audio tasks that have been intensively explored in protein generation of unaligned sequences.

Deep generative models are models for learning high-dimensional data distributions and generating novel data samples indistinguishable from the true data. Subsequently, DGMs are promising for synthetic protein design. For example, autoregressive models (i.e., language or audio generative models) have no dependency on sequence alignments, allowing these models to learn and generate novel sequences with high variability and diverse lengths. However, one major limitation of autoregressive models is the lack of ability to infer meaningful representations or conditional information (e.g., latent vectors).

In contrast, variational autoencoders (VAEs) can infer a latent space and generate novel data indistinguishable from a true data distribution. These models have been shown to effectively predict single-mutant effects, infer a homologous family's phylogeny through the latent space, and diversify synthetic AAV capsids. See Sam Sinai, Nina Jain, George M Church, and Eric D Kelsic, “Generative AAV capsid diversification by latent interpolation,” bioRxiv, 2021, incorporated herein by reference in its entirety. While these models can infer a biologically meaningful latent space, they struggle to implement powerful and expressive decoders (i.e., generators) because VAEs are prone to posterior collapse. See Shengjia Zhao, Jiaming Song, and Stefano Ermon, “Infovae: Balancing learning and inference in variational autoencoders,” in Proceedings of the aaai conference on artificial intelligence, volume 33, pp. 5885-5892, 2019; Xi Chen, Diederik P Kingma, Tim Salimans, Yan Duan, Prafulla Dhariwal, John Schulman, Ilya Sutskever, and Pieter Abbeel, “Variational lossy autoencoder, “arXivpreprint arXiv:1611.02731, 2016; Aaron Van Den Oord, Oriol Vinyals, et al., “Neural discrete representation learning,” Advances in neural information processing systems, 30, 2017; and Zichao Yang, Zhiting Hu, Ruslan Salakhutdinov, and Taylor Berg-Kirkpatrick, “Improved variational autoencoders for text modeling using dilated convolutions,” in International Conference on Machine Learning, PMLR, 2017, each incorporated herein by reference in their entirety. Therefore, VAEs struggle with incorporating autoregressive decoders for generating variable-length sequences and inferring alignment-free homologous protein datasets.

### SUMMARY

An aspect of the present disclosure is a method of generating protein sequences, the method including encoding, using a dilated convolutional encoder, a plurality of input protein sequences onto a latent space distribution; and decoding, using a decoder employing dilated causal convolutions, the latent space distribution to generate new protein sequences different from the input protein sequences.

A further aspect of the present disclosure is a method of generating protein sequences using a system including an encoder coupled to an autoregressive generator, and having been trained with a loss function that comprises reconstruction loss and a mutual information maximization term, the method including encoding, using the encoder, a plurality of input protein sequences onto a latent space distribution; and decoding, using the autoregressive generator, the latent space distribution to generate new protein sequences different from the input protein sequences.

A further aspect of the present disclosure is a method of training a system for generating protein sequences, the system including an encoder that encodes a plurality of input protein sequences onto a latent space distribution, and an autoregressive generator that decodes the latent space distribution to generate new protein sequences different from the input protein sequences, the method including training the system with a loss function that comprises reconstruction loss and a mutual information maximization term.

A further aspect of the present disclosure is a system for generating protein sequences, including an encoder configured to encode a plurality of input protein sequences onto a latent space distribution; and an autoregressive generator configured to decode the latent space distribution to generate new protein sequences different from the input protein sequences, in which the system is trained with a loss function that includes reconstruction loss and a mutual information maximization term.

A further aspect of the present disclosure is a non-transitory computer-readable medium storing a program that, when executed by processing circuitry, causes the processing circuitry to perform a method of generating protein sequences using a system including an encoder coupled to an autoregressive generator, and having been trained with a loss function that includes reconstruction loss and a mutual information maximization term, the method including encoding, using the encoder of a variational autoencoder, a plurality of input protein sequences onto a latent space distribution; and decoding, using the autoregressive generator, the latent space distribution to generate new protein sequences different from the input protein sequences.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 is a flowchart for a method of generating novel protein sequences or reconstructing input protein sequences;

FIGS. 2 to 8 are flowcharts for an inference method of generating protein sequences;

FIGS. 9 to 15 are flowcharts for a training method of generating protein sequences;

FIG. 16 is a schematic diagram of an architecture for a combination of the InfoMax Variational autoencoder with convolutional encoder and a Wavenet-based decoder;

FIG. 17 is a schematic diagram of a decoder in more detail in one embodiment;

FIG. 18 is a network diagram of a stack of dilated convolution layers;

FIG. 19A is a plot of latent space embeddings learned over aligned sequences and FIG. 19B is a plot of latent embeddings learned over unaligned sequences;

FIG. 20A is a plot of latent embeddings when the model is trained on input sequences with multiple sequence alignment, while FIG. 20B is a plot of latent embeddings of natural and synthetic sequences when using a model trained on unaligned input data;

FIG. 21 is a display for predicted structures of four synthetically designed SH3 sequences;

FIG. 22 is a display for predicted structures of four synthetically designed CM sequences;

FIG. 23A is a panel that compares the generated sequences in terms of sequence length and similarity to the nearest natural SH3 homolog, while FIG. 23B is a panel that compares the generated sequences' lengths and similarity to the nearest natural CM homolog;

FIGS. 24A and 24B show possible 2D projections of the SH3 latent space;

FIGS. 25A, 25B, 25C, and 25D show possible 2D projections of the CM latent space;

FIG. 26 is a block diagram of a system environment in which a method according to the present disclosure can be performed; and

FIG. 27 is a block diagram of a computer system.

### DETAILED DESCRIPTION

A problem that occurs when combining autoregressive models and VAEs for protein design and fitness prediction is that VAEs are prone to posterior collapse when implemented with an expressive decoder. A disclosed solution is to use a VAE with a WaveNet-based decoder. WaveNet-based generators can provide inexpensive training time and computation cost relative to recurrent neural networks (RNNs) and avoid vanishing gradients, because WaveNet leverages dilated causal convolutions. In addition, to avoid posterior collapse, the disclosed model implements and adapts an Information Maximizing VAE (InfoVAE) loss objective, instead of a standard Evidence Lower Bound (ELBO) training objective, to a semi-supervised setting with an autoregressive reconstruction loss. Further, in one embodiment the disclosed model is extended from unsupervised to a semi-supervised learning paradigm for fitness prediction tasks enabling effective prediction of fitness from sequences alone.

In one embodiment, the disclosed model's performance is benchmarked on FLIP and TAPE datasets for protein function prediction. To illustrate the model's performance for protein design, the model is trained on unaligned homologous sequence libraries of the SH3 domain and AroQ Chorismate mutase enzymes, for example. Then, the trained model is deployed to generate novel (variable-length) sequences that are computationally predicted to fold into native structures and possess natural function. The results demonstrate that combining a semi-supervised InfoVAE model with a WaveNet-based generator provides a robust framework for functional prediction and generative protein design, without requiring multiple sequence alignments.

As mentioned above, in order to successfully combine VAEs with autoregressive generators and overcome posterior collapse, a disclosed embodiment incorporates an Information Maximizing (InfoMax) loss objective instead of the common ELBO training objective. See Samuel R Bowman, Luke Vilnis, Oriol Vinyals, Andrew M Dai, Rafal Jozefowicz, and Samy Bengio, “Generating sentences from a continuous space,” arXiv preprint arXiv:1511.06349, 2015, incorporated herein by reference in its entirety. The InfoMax loss is similar to ELBO. However, prefactor weights are introduced to motivate better inference and regularization. Further, a mutual information maximization term is introduced for explicitly encouraging high mutual information between the input vectors and latent space embeddings. A WaveNet-based autoregressive generator is implemented for the decoder that avoids vanishing or exploding gradients by leveraging dilated causal convolutions. See Aaron van den Oord, Sander Dieleman, Heiga Zen, Karen Simonyan, Oriol Vinyals, Alex Graves, Nal Kalchbrenner, Andrew Senior, and Koray Kavukcuoglu, “Wavenet: A generative model for raw audio,” arXiv preprint arXiv:1609.03499, 2016, incorporated herein by reference in its entirety.

Previously, models have been developed that combine VAEs with dilated causal convolutions as the decoder component for text generation. However, this approach does not carry over to protein design and fitness prediction. Disclosed embodiments expand this modeling approach by incorporating an InfoMax loss objective for improving amortized inference and avoiding posterior collapse. In one embodiment, the convolutions are much faster than recurrent networks during training time, offer superior inference of long-range correlations, and are computationally lighter-weight than standard convolutional filters. The InfoVAE can infer biologically meaningful latent spaces while incorporating an expressive autoregressive generator. The InfoVAE training objective is extended to a semi-supervised learning paradigm for fitness landscape prediction.

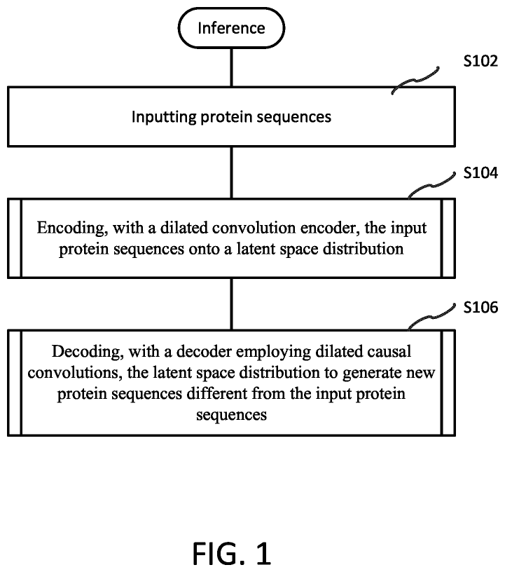

FIG. 1 is a flowchart of a method of generating novel protein sequences or reconstructing input protein sequences. The method begins by, in S102, inputting protein sequences. In S104, a dilated convolution encoder encodes the input protein sequences onto a latent space distribution z0. The latent space z0 may be approximated as a tractable Gaussian distribution. In S106, a decoder employing dilated causal convolutions decodes the latent space distribution to generate new protein sequences different from the input protein sequences, or reconstructs the input protein sequences. A protein's 3D structure can be predicted from the output protein sequence. In one embodiment, AlphaFold2 is a tool that is used to predict the protein 3D structure.

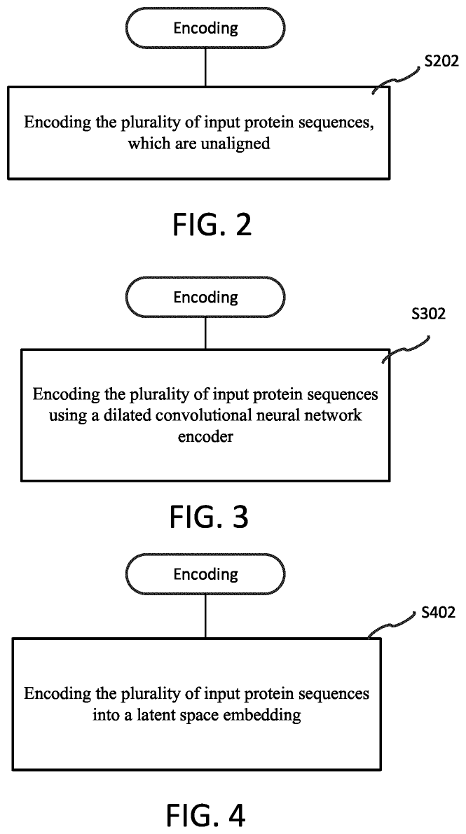

Regarding FIG. 2, in step S202, the encoding step can include encoding the plurality of input protein sequences, which are unaligned.

Regarding FIG. 3, in step S303, the encoding step can include encoding the plurality of input protein sequences using a dilated convolutional neural network encoder.

Regarding FIG. 4, in step S403, the encoding step can include encoding the plurality of input protein sequences into a latent space embedding.

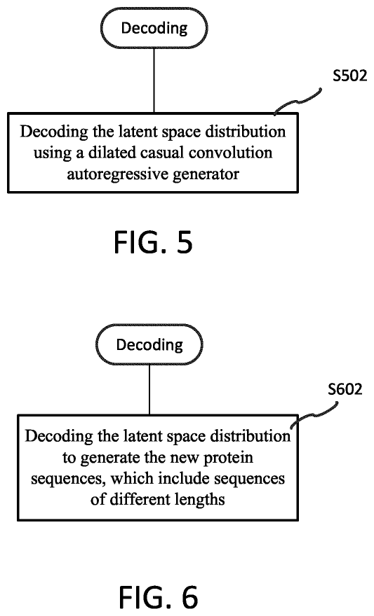

Regarding FIG. 5, in step S502, the decoding step can include decoding the latent space distribution using a dilated casual convolution autoregressive generator as the autoregressive generator.

Regarding FIG. 6, in step S602, the decoding step can include decoding the latent space distribution to generate the new protein sequences, which include sequences of different lengths.

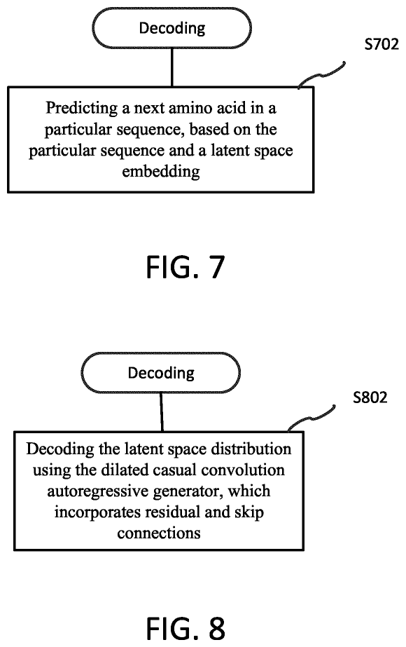

Regarding FIG. 7, in step S702, the decoding step can include predicting a next amino acid in a particular sequence, based on the particular sequence and a latent space embedding.

Regarding FIG. 8, in step S802, the decoding step can include decoding the latent space distribution using the dilated casual convolution autoregressive generator, which incorporates residual and skip connections.

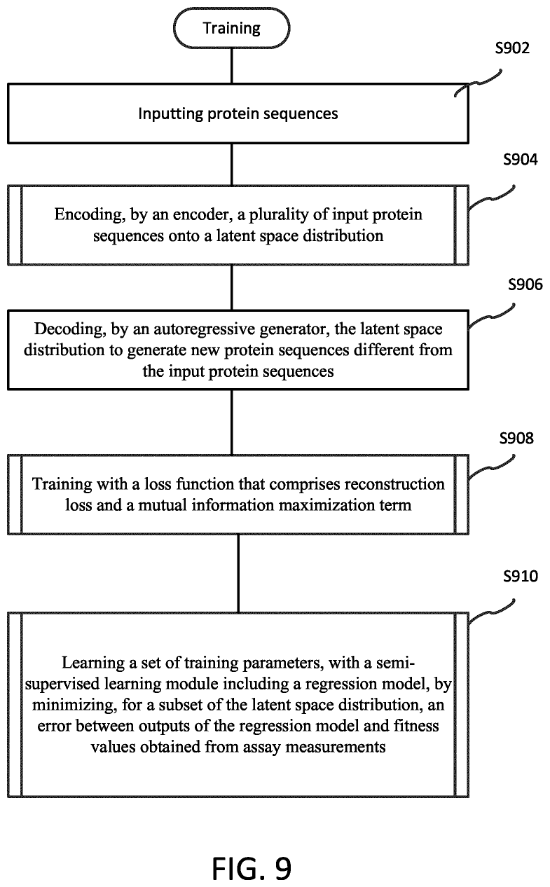

FIG. 9 is a flowchart for a method of training a system for generating protein sequences. The method includes, in step S902, inputting protein sequences. In step S904, the encoder encodes a plurality of input protein sequences onto a latent space distribution. In step S906, an autoregressive generator decodes the latent space distribution to generate new protein sequences different from the input protein sequences. In S908, the system is trained with a loss function that includes reconstruction loss and a mutual information maximization term. In step S910, a set of training parameters of a regression model are learned in a semi-supervised learning module by minimizing, for a subset of the latent space distribution, an error between outputs of the regression model and fitness values obtained from assay measurements.

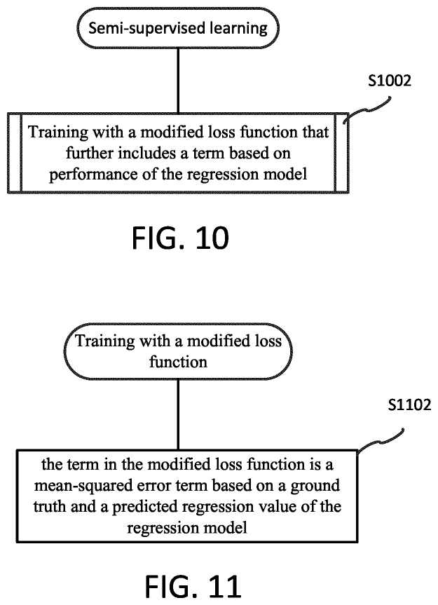

Regarding FIG. 10, in step S1002, the semi-supervised learning module trains the system with a modified loss function that further includes a term based on performance of the regression model.

Regarding FIG. 11, in step S1102, in training the system with the modified loss function, the term in the modified loss function is a mean-squared error term based on a ground truth and a predicted regression value of the regression model.

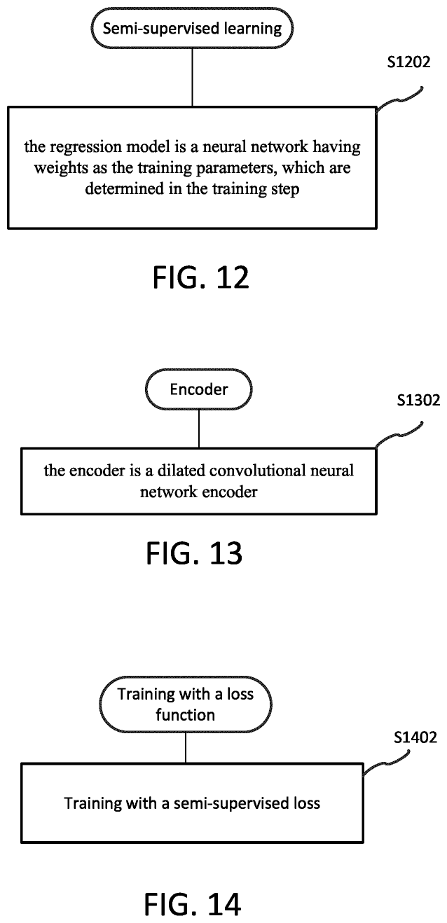

Regarding FIG. 12, in step S1202, in the semi-supervised learning module, the regression model is a neural network having weights as the training parameters, which are determined in the training step.

Regarding FIG. 13, in step S1302, the encoder is a dilated convolutional neural network encoder.

Regarding FIG. 14, in step S1402, the training step can include training the system with the loss function, which further includes a semi-supervised loss.

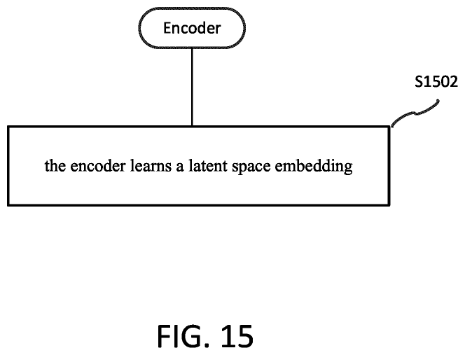

Regarding FIG. 15, in step S1502, the encoder learns a latent space embedding.

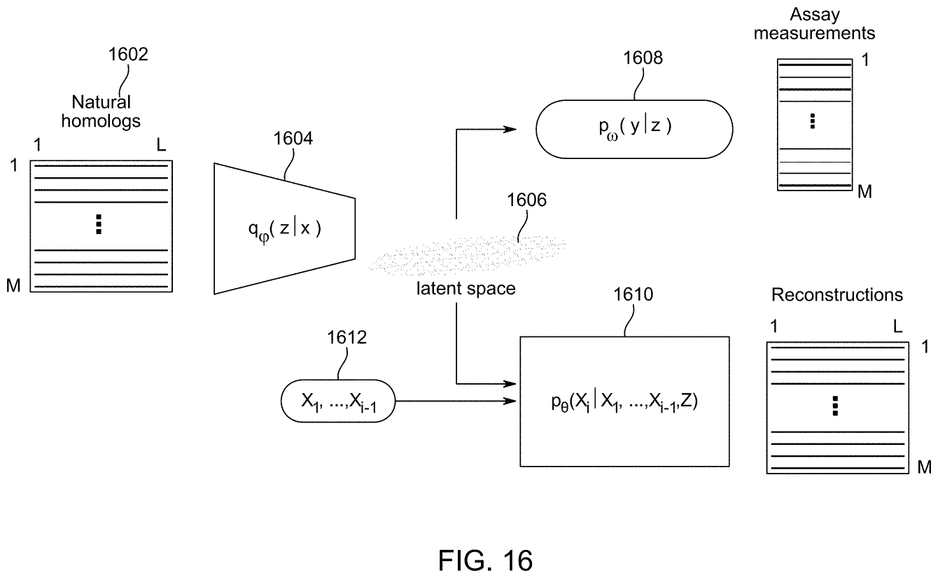

FIG. 16 is a schematic diagram of an architecture for a combination of the InfoMax Variational autoencoder with convolutional encoder and a Wavenet-based decoder. The proteins sequences, either aligned or unaligned, (natural homologs) 1602 are embedded onto a lower-dimensional latent space 1606 using a dilated convolutional neural network encoder q_{ϕ}(z|x) 1604. The decoder (i.e., generator) p_{θ}(x|z) 1610 is a conditional WaveNet-based architecture, which samples from the latent space 1606 and predicts amino acid residues while conditioning on previous amino acids p_{θ}(x|z)=p(x₀|z)Πᵢ₌₁p(xᵢ|x_{<i}z) 1610. Generally, when using a dilated causal convolution and predicting the next amino acid residue, teacher forcing is used, which leverages the true labeled amino acids for previous conditional amino acids. Since the decoder 1610 is an autoregressive generator, variable length sequences can be designed. In addition, this model can be extended to a semi-supervised paradigm such that a discriminator neural network (a simple fully connected regression model; p_{w}(y|z)) 1608 samples the latent space 1606 and predicts fitness or function measurements. In the semi-supervised paradigm, the discriminative and generative losses are learned together.

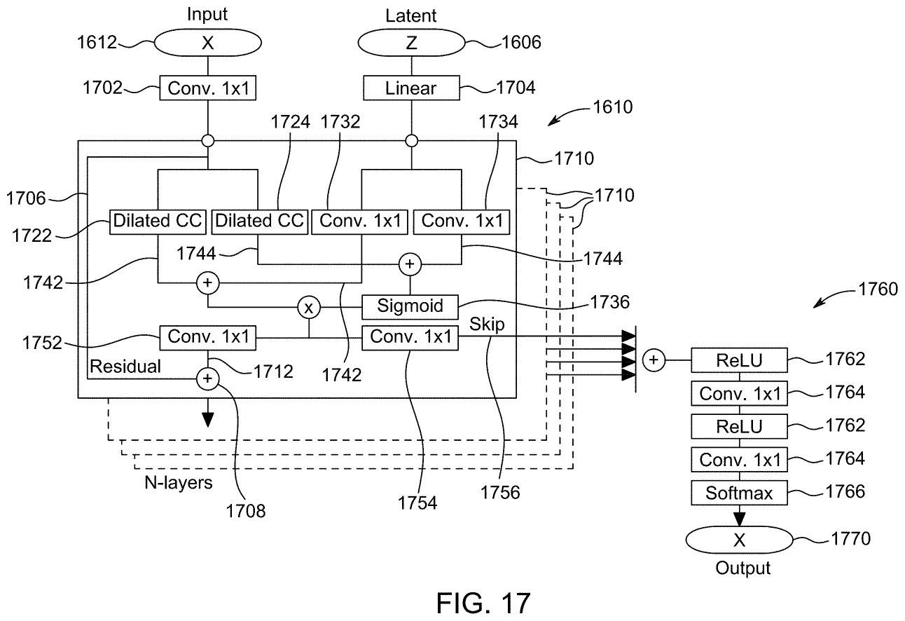

FIG. 17 is a schematic diagram of the decoder 1610 in more detail. In the decoder 1610, the conditional probability distribution is modelled by a stack of convolutional layers. There are no pooling layers in the decoder network, and the output of the model has the same time dimensionality as the input. The model outputs a categorical distribution over the next value xt with a softmax layer, and is optimized to maximize the log-likelihood of the data with respect to the parameters.

The original WaveNet model includes causal convolutions. By using causal convolutions, the model cannot violate the ordering in which the data is modeled. However, one of the problems of causal convolutions is that they require many layers, or large filters to increase the receptive field. The conditional WaveNet-based architecture 1610 uses dilated convolutions to increase the receptive field by orders of magnitude, without greatly increasing the computational cost.

The WaveNet decoder shown in FIG. 17 has two independent inputs: the first input 1612 accepts aligned or unaligned protein sequences (“x”), and the second input 1606 takes latent vectors “z.” The amino acids are represented as one-hot encoded for the input protein sequences 1612 (“x”). Before entering the WaveNet module 1610, the convolution layer 1702 with a 1×1 kernel maps these one-hot encoded representations as more meaningful continuous representations. Similarly, before entering the WaveNet module 1610, the latent vectors 1606 (“z”) are mapped from a lower-dimensional vector (1×dim(z)) to a latent vector with the same size as the protein sequence using a linear layer 1704. The variable dim(z) is a hyperparameter chosen by the machine learning practitioner. After the input data has been transformed by the convolution 1×1 1702 and linear layer 1704 and entered the WaveNet module 1610, the protein sequence data is copied and passed through a “Residual” connection 1706. The protein sequences passed through the “Residual” connection 1706 are added 1708 at the end of each WaveNet layer 1710 (N layers) with the final output 1712 after going through many operations. These “Residual” connections 1706 allow easier gradients to flow through deep networks and avoid vanishing gradients. Before adding the final output 1712 with “Residual connection” 1706 at each WaveNet layer 1710, the protein input data 1612 “x” is passed through two independent dilated causal convolution (CC) operations 1722, 1724. The left dilated CC 1722 corresponds to the “signal” operation (linear function 1704), and the right dilated CC 1724 corresponds to the “gate” operation (sigmoid function 1726).

Similarly, simple 1×1 convolution layers 1732, 1734, i.e. instead of dilated causal convolutions, are used to transform the latent input vectors 1606. These transformed latent vectors are passed through two independent operations: one operation corresponds to the “signal” path 1742, and the second corresponds to the “gate” path 1744. After the protein input 1612 and latent data 1606 are passed through the signal path 1742, these output transformations are only summed together and are not followed by a nonlinear activation function—illustrated by “(+).” While the gate paths 1744 sum the output representation for the protein and latent input data, a nonlinear activation function, specifically the sigmoid function 1736, further transforms the output. After both signal 1742 and gate 1744 paths are complete and ready to merge the protein and latent input data, the final result of the gate and signal is multiplied together—illustrated by “(*).” Then, this final multiplied output is independently passed through two more convolution 1×1 operations 1752, 1754, where one result is added back to the “Residual” connection 1706, and the second output is added and passed through a “Skip” connection 1756. The output which went through the “Residual” path 1712 is given to the next WaveNet layer 1710 of the WaveNet module 1610 as the input protein sequence data 1612 “x” while the latent input data 1606 for the next layer remains the same. For the output of the “Skip” connection 1756, the output transformation is accumulated (summed “(+)”) with the output transformation of each WaveNet layer 1710 implemented in the WaveNet module 1610. After the cumulation is complete with each WaveNet layer 1710, the transformation is passed through a “WaveNet top head 1760,” which passes the data through ReLU activation functions 1762 and Convolution 1×1 layers 1764 until it reaches a Softmax operation 1766 that reconstructs the input sequence to provide an output protein sequence 1770.

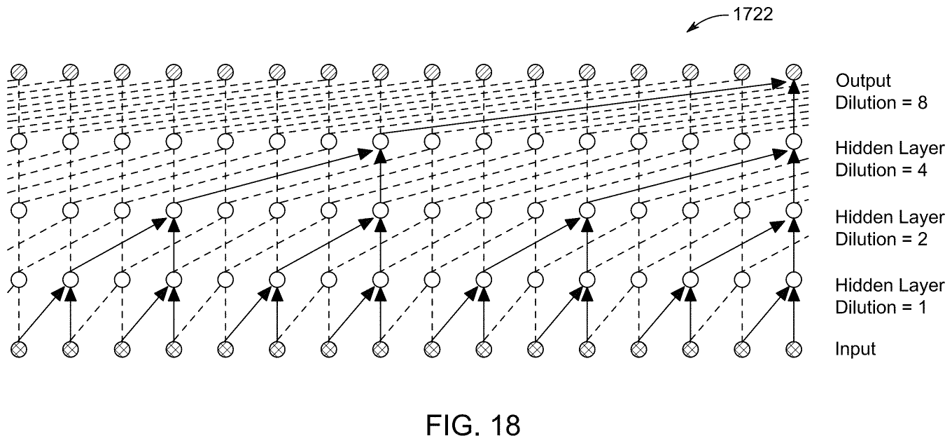

FIG. 18 is a network diagram of a stack of dilated convolution layers 1722. A dilated convolution is a convolution where the filter is applied over an area larger than its length by skipping input values with a certain step, and is equivalent to a convolution with a larger filter derived from the original filter by dilating it with zeros, but is significantly more efficient. A dilated convolution effectively allows the network to operate on a coarser scale than with a normal convolution. As a special case, dilated convolution with dilation 1 yields the standard convolution. FIG. 18 depicts dilated causal convolutions for dilations 1, 2, 4, and 8. Stacked dilated convolutions enable networks to have very large receptive fields with just a few layers, while preserving the input resolution throughout the network as well as computational efficiency.

Integrating InfoVAE with a WaveNet-Based Generator

Traditional Variational Autoencoders (VAEs) are prone to posterior collapse or poor amortized inference when implementing expressive decoders (e.g. autoregressive generators). Here, a VAE model is implemented to overcome posterior collapse and improve variational inference when implementing a WaveNet-based autoregressive decoder. In one embodiment, he unsupervised loss function is the following:

[symbol]

_{US}=ξ [symbol] z˜q_{ϕ}(z|x)[log p_{θ}(x|z)]−(1−α) [symbol] _{KL}(q_{θ}(z|x)∥p(z))−(α+λ−1) [symbol] _{MMD}(q_{ϕ(z)}∥p(z)) (1)
where p_{θ}(x|z) 410 is the decoder model, [symbol] _{KL} is the Kullback-Leibler divergence between the variational posterior approximation q_{ϕ}(z|x) and normal prior distribution p(z). The third term [symbol] _{MMD} is the max-mean discrepancy (MMD), which helps penalize the aggregated posterior distribution and improves amortized inference. In this disclosure, an autoregressive decoder 410 is incorporated (WaveNet-based architecture), where p_{θ}(x|z)=p_{θ}(x₀|z) Πᵢ₌₁p_{θ}(xᵢ|x_{<i},z). The MMD divergence term becomes [symbol] _{MMD}= [symbol] _{z,z′˜p(z) p(z′)}[k(z,z′)]−2 [symbol] _{z,z′-q(z),p(z′)} [k(z, z′)+ [symbol] _{z,z′˜q(z),q(z′)} [k(z, z′)], where k(·, ·) is a positive definite kernel and [symbol] _{DMMD}=0 if and only if p(z)=q(z). The Gaussian kernel k(z, z′)=e^{(z-z)2/σ}^{²} is chosen as the characteristic kernel k(·, ·), and σ is a hyperparameter defining the bandwidth of the Gaussian kernel. The prefactor loss weights ξ, α, and λ scales the contribution of the reconstruction loss, weighs the mutual information between x and z, and scales the penalization of MMD divergence.

Extending Generative Model to a Semi-Supervised Paradigm

The unsupervised WaveNet VAE is extended to a semi-supervised learning paradigm for fitness landscape prediction. The main motivation of using a semi-supervised approach is based on the idea that latent representations z can be more informative for predicting y when also used for reconstructing x. In addition, semi-supervised learning is beneficial when labels are scarce, and unlabeled data is abundant, which is generally the case for protein design over large unlabeled sequence databases for which a small fraction of sequences are labeled with functional assays. The semi-supervised training objective is the following:

[symbol]

_{SS}= [symbol] _{US}+γ [symbol] [log p_{ω}(y|z)] (2)
where p_{w}(y|z) is a regression model (a simple fully connected neural network) parameterized with training parameters ω. In practice, the mean-squared error objective ½ |y−ŷ|² is minimized, where y and ý is the ground truth and predicted regression value. The (x,y)∈ [symbol] _{L} denotes that the samples which are fed through the supervised branch are only sequences x with assay measurements y.

Training and Hyperparameter Optimization

In one embodiment, during training, ξ, α, λ, γ, and σ are set to 1, 0.95, 2, 1, and \sqrt{dim(z)}. Then, hyperparameter optimization is conducted over the latent space dimensions z∈[1,20] for each fitness landscape prediction task. The optimal latent space dimension was chosen based on minimizing the negative-log likelihood and maximizing the Spearman ρ score on the validation set. In general, the prefactor loss weights can be optimized as well. The optimization algorithm used in this study was Adam with a learning rate of 1e−4. See Diederik P Kingma and Jimmy Ba, “Adam: A method for stochastic optimization,” arXiv preprint arXiv:1412.6980, 2014, incorporated herein by reference in its entirety. For fitness prediction tasks, the number of epochs was set to 2000, and early stopping was only implemented if p reaches a value of 0.99 on the validation set. For simplicity, the minibatch size is set across all fitness prediction tasks to 256 samples, but this too can be optimized.

Results: Protein Design

To illustrate the advantage of combining VAEs with an autoregressive WaveNet decoder, the model was trained on unaligned homologous datasets, in one embodiment. To show that the model can handle unaligned homologous sequences, the latent embeddings are compared and learned representations between unaligned and aligned sequence datasets.

Description of the Homolog Family Datasets

The disclosed model is trained and generated novel sequences from two homologous protein families: Src homologous 3 domains (SH3) and AroQ chorismate mutase (CM) enzymes. The SH3 family consists of many paralogs, which are homologous sequences that diverge due to duplication events. Since the gene is duplicated in the genome, new selective pressure can act on the duplicated gene, and subsequently, paralogs can acquire new functions. The SH3 dataset size is 5611 sequences, consisting primarily of proteins found in the fungal kingdom. Of the 5611 sequences in the basebase, 4664 are labeled with functional assay measurements for osmosensing capabilities. The CM dataset differs from the SH3 dataset because all of proteins are orthologs, which are homologous sequences that diverge due to speciation events instead of duplication events. This means most of the natural homologous CM sequences have a similar catalytic function. In addition, the CM dataset has two sets of proteins—one set corresponds to the natural homologs (1130 sequences), while the second set corresponds to synthetic designs (1618 sequences) produced using a direct coupling analysis (DCA) model that explicitly considers only pairwise epistasis. See William P Russ, Matteo Figliuzzi, Christian Stocker, Pierre Barrat-Charlaix, Michael Socolich, Peter Kast, Donald Hilvert, Remi Monasson, Simona Cocco, Martin Weigt, et al., “An evolution-based model for designing chorismate mutase enzymes,” Science, 369(6502):440-445, 2020, incorporated herein by reference in its entirety. All sequences have been functionally assayed for CM catalytic function.

Latent Space Interpretation

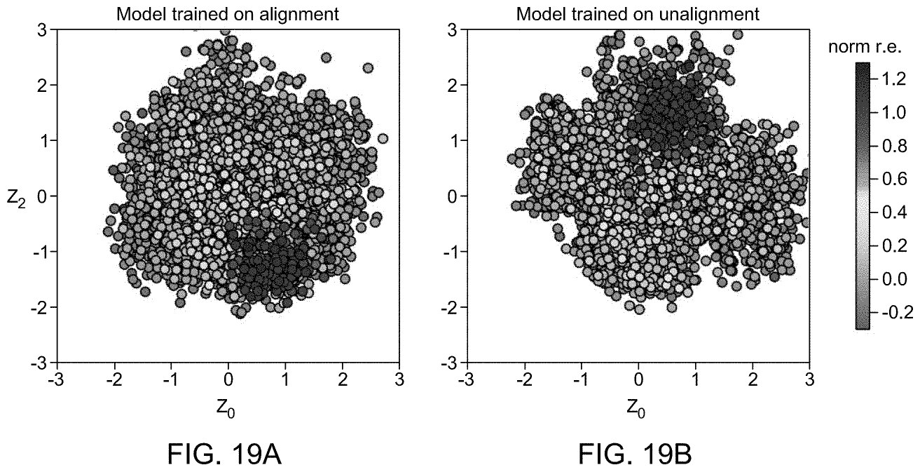

FIGS. 19A and 19B presents the latent space embeddings of the SH3 dataset produced by unsupervised training (Eqn. 1) of the model operating on aligned (FIG. 19A) and unaligned (FIG. 19B) training data. The embedded points may be colored according to an experimental select-seq assay that reports a proxy measure for osmosensing function termed a normalized relative enrichment (n.r.e.) score. A n.r.e.=1 corresponds to activity comparable to the wild type SH3; a n.r.e.=0 corresponds to activity commensurate with a null gene. In both, the aligned and unaligned latent space embeddings are observed with strong clustering of the highly active osmosensing sequences with n.r.e. scores of 1.0. This demonstrates that the fully unsupervised model has learned biologically meaningful representations of the sequence ensemble separating orthologs (sequences with osmosensing function; red) from paralogs (sequences lacking osmosensing activity; blue).

Referring to FIGS. 19A and 19B, the figures show a plot of the latent space embeddings of the natural SH3 homolog library, consisting of various different paralog groups and including the Sh0l paralog group. The colorbar represents in vivo fitness and indicates whether a SH3 homolog can rescue osmosensing functionality in S. cerevisiae. The latent spaces learned over (FIG. 19A) aligned and (FIG. 19B) unaligned sequences both provide a good separation of the high activity orthologs (red) and low activity paralogs (blue). Importantly, both models were trained using an unsupervised learning approach that was not exposed to functional assay measurements, indicating the generative model is able to learn meaning representation for designing function solely from unlabeled sequences.

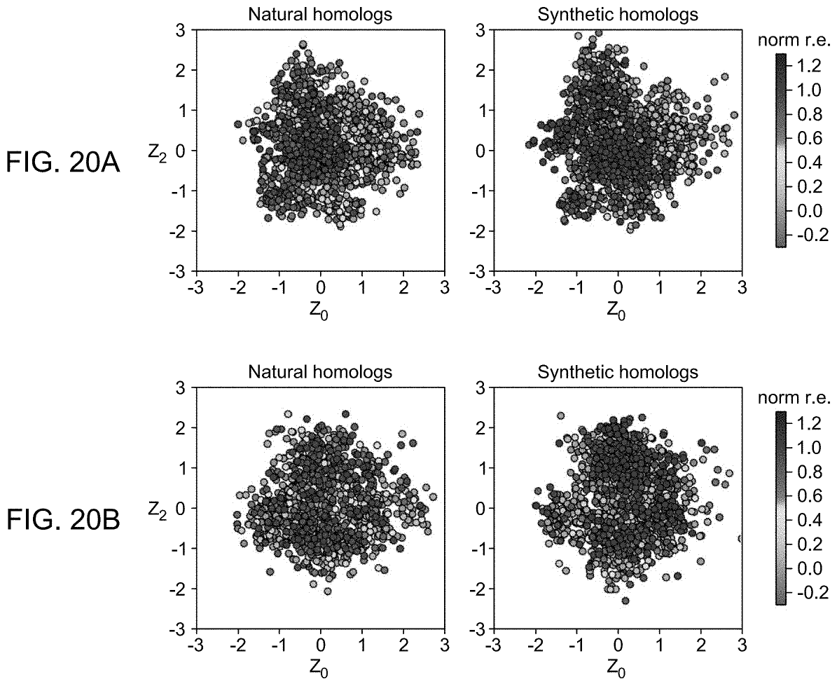

FIGS. 20A and 20B illustrate the aligned and unaligned latent space embeddings for the CM dataset. When training the model on the CM dataset, the training set contains only the natural homologs, while the validation set contained the synthetic designs. As was observed for the SH3 data, the unsupervised model learns a meaningful latent space embedding of the training data in which an emergent clustering and gradient are observed in catalytic activity, again measured by a select-seq assay that provides a n.r.e. score. The model is able to generalize quite well by embedding the synthetic designs onto the latent space and clustering high fitness embeddings into a region of the space.

Referring to FIGS. 20A and 20B, those figures show a plot of the latent embeddings of both training (natural CM homologs) and validation (synthetic CM homologs) set. FIG. 20A shows the latent embeddings when the model was trained on input sequences with a multiple-sequence alignment, while FIG. 20B shows the latent embeddings of natural and synthetic CM sequences when using a model trained on unaligned input data. Since the CM training dataset consists of only orthologs, the latent space is more scattered in terms of high fitness embeddings (n. r.e.). However, the overall latent space retains the Gaussian structure and learns to cluster some high fitness regions regardless of whether the model was trained on aligned or unaligned input data.

Generation of Novel Variable Length Sequences with Structure Prediction

To illustrate the practical advantage of using an autoregressive decoder, the model trained on aligned and unaligned sequence data is compared for both the SH3 and CM datasets. Novel sequences are generated by randomly sampling points within the latent spaces and decoding these through the WaveNet generator to produce novel protein sequences. For the SH3 system, 5611 novel sequences are sampled and generated, while for the CM system, 1130 novel sequences are generated. To check whether the generated sequences fold into a proper tertiary structure, AlphaFold2 was used to predict structures of four sequences for both the SH3 and CM task: the shortest generated sequence, the longest generated sequence, and two randomly selected sequences (Tables 2 and 3).

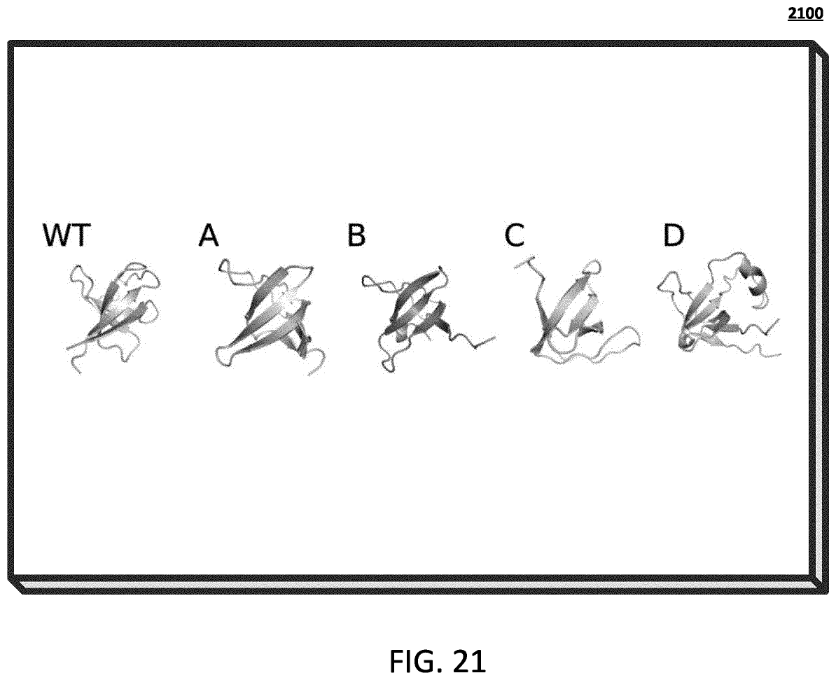

With the SH3 task, the structure predictions of the generated sequences can be displayed in a display device 2100 along with the wild-type SH3 domain (PDB: 2VKN) as shown in FIG. 21. All four sequences are predicted to have a very similar tertiary structure as the wild-type, even though the sequence similarity to the nearest natural SH3 domain lies between only 41-43%. (A sequence is defined based on similarity to the nearest natural homolog as

$$1 - \frac{η}{{L}_{max}} ,$$

where η and Lₘₐₓ are the minimum Hamming distance and longest protein sequence within the natural homolog library). Interestingly, the longest SH3 domain among the generated pool is predicted to acquire an alpha helix, which was originally a hairpin loop on the WT structure (D). It is important to note that this sequence is 11 amino acids longer than the wild-type SH3 domain (Table 2), potentially allowing the generative model to extrapolate in terms of design.

TABLE 1
GB1 performance comparison to current baseline scores
(metric: Spearman correlation).

| | low-vs-high | 1-vs-rest | 2-vs-rest | 3-vs-rest |
|---|---|---|---|---|
| Architecture | ρ | ρ | ρ | ρ |
| ESM-1b | 0.59 | 0.28 | 0.55 | 0.79 |
| ESM-1b | 0.13 | 0.32 | 0.36 | 0.54 |
| ESM-1v | 0.10 | 0.32 | 0.32 | 0.77 |
| Ridge | 0.34 | 0.28 | 0.59 | 0.76 |
| CNN | 0.51 | 0.17 | 0.32 | 0.83 |
| Levenshtein | −0.1 | −0.17 | 0.16 | 0.01 |
| BLOSUM62 | −0.13 | 0.15 | 0.14 | 0.91 |
| Our model | 0.42 | 0.28 | 0.61 | 0.87 |

See Christian Dallago, Jody Mou, Kadina E. Johnston, Bruce J. Wittmann, Nicholas Bhattacharya, Samuel Goldman, Ali Madani, and Kevin K. Yang, “Flip: Benchmark tasks in fitness landscape inference for proteins,” bioRxiv, 2022, incorporated herein by reference in its entirety.

TABLE 2
Primary structure of the
generated SH3 proteins in FIG. 21

| Label | Primary structure | AA length |
|---|---|---|
| WT | DDNFIYKAKALYPYDADDDDAYEISFE | 70 |
| | QNEILQVSDIEGRWWKARRANGETGII | |
| | PSNYVQLIDGPEEMHR | |
| A | ASTLFYARALYDYTAQGDDELSVAEGD | 61 |
| | LLYVLERDDDGWWKAEKDGGAGGEPAE | |
| | PIELLNP | |
| B | APAVETATALYDYEAQADGDLSFSFGD | 59 |
| | RITIVERTNSDDWWYGRNNRGEFGFFP | |
| | ANYVE | |
| C | APGGVYAVVLYDFDANGDDEVDVKEGE | 51 |
| | ELVILDRSNPEWFVAKNPATGEPV | |
| D | APPKKVARALYDFTAEGDDELDVKEGD | 81 |
| | VVLVLEKDDGYWLVVKDDGTGGGPVVW | |
| | LQSCYAVTDSSGLVPVSYVEIVPASTT | |

TABLE 3
Primary structure of the generated CM proteins

| | | AA |
|---|---|---|
| Label | Primary structure | length |
| WT | PLLALREKISALDEKLLALLAERRELAVEVGKAKLL | 95 |
| | SHRPVRDIDRERDLLERLITLGKAHHLDAHYITRLF | |
| | QLIIEDSVLTQQALLQQHLNKIN | |
| A | SDLEELREEIDQIDRQIIDLLAERMKRVREVGQYKI | 91 |
| | SKGGPVFDPPREAEVIERLRRLAAAPLGDPERVAAL | |
| | LRRLIEESVLDQLDEELVK | |
| B | SDLEELREEIDQIDRQIDELLAERLKLVAEVGEYKA | 90 |
| | SIGLPVYDPKREAQVLDRLRELAKNAGLDPEFAELF | |
| | LDFVIAEIIRHHEAIQNK | |
| C | SDLEELREEIDQIDRQIIDLLNERMKIVREVGEYKI | 50 |
| | SKGLPVYDPEREKQ | |
| D | SDAELLELRRRQIDIDDARLELLAERRRRVAEVAAL | 96 |
| | KKLANGLPRRFRREEAVLLKRLSRAAEPPGPADVAA | |
| | LLRRLIRAAARAQAAEAFAERRRL | |

Regarding FIG. 21, AlphaFold2 predicted structures of four synthetically designed SH3 sequences. The first column corresponds to the WT Sho1^{SH3} domain in S. cerevisiae (PDB: 2VKN) which has a length of 70 amino acids. The next four columns (A-D) corresponds to design sequences with variable lengths, where the A, B corresponds to randomly generated sequences and C, D corresponds to the shortest, longest generated SH3 domains. The primary structure and sequence length for these proteins are shown in Table 2.

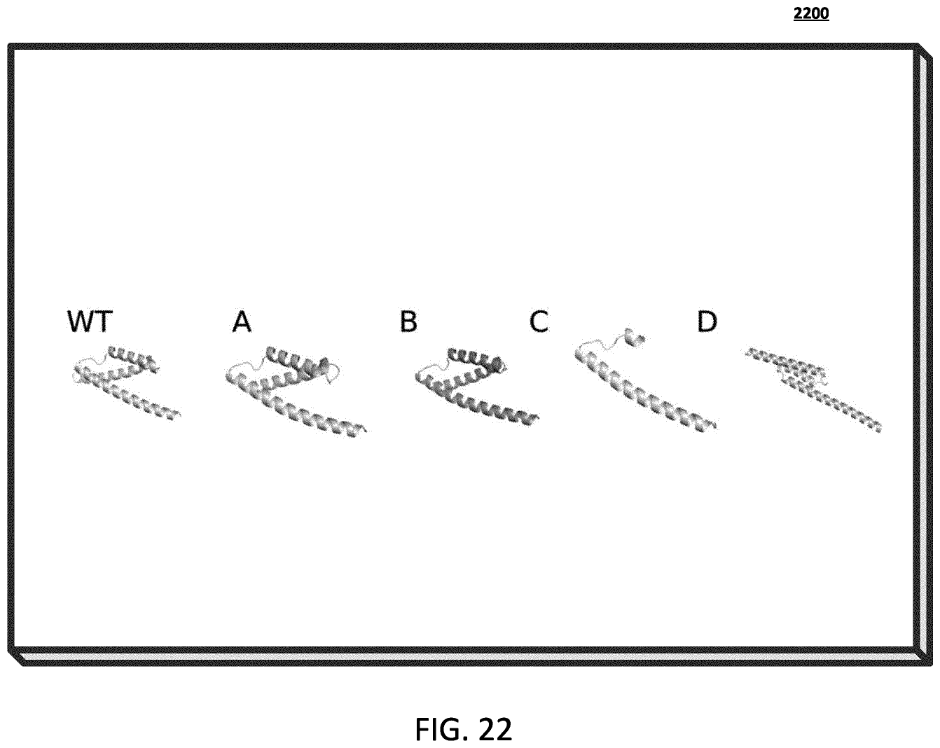

With the CM task, the two randomly sampled sequences from the generated pool (A,B in FIG. 22) have a very similar tertiary structure to the wild-type AroQ CM monomer in Escherichia coli (PDB: 1ECM). The shortest generated CM sequence is missing a whole alpha helix and a significant portion of a second alpha helix compared to the wild-type, but is still predicted to fold into a stable tertiary structure even though it is 45 amino acids shorter than the WT sequence (Table 3). In D of FIG. 22, the longest generated CM sequence seems to maintain all three alpha helices, but these helices are predicted to be longer and straighter than the wild-type helices. The four CM generated sequences have a sequence similarity to the nearest natural CM homolog of 0.18-0.21%.

Regarding FIG. 22, that figure shows a display 2200 of AlphaFold2 predicted structures of four synthetically designed CM sequences. The first column corresponds to the WT AroQ Chorismate mutase (CM) enzyme in Escherichia coli (PDB: 1ECM), while the next two columns (A, B) corresponds to design sequences randomly sampled from the generated pool of CM sequences. Columns (C, D) correspond to the remaining two generated sequences that are shortest and longest sequence within the generated pool. The primary structure and sequence length for these proteins are shown in Table 3.

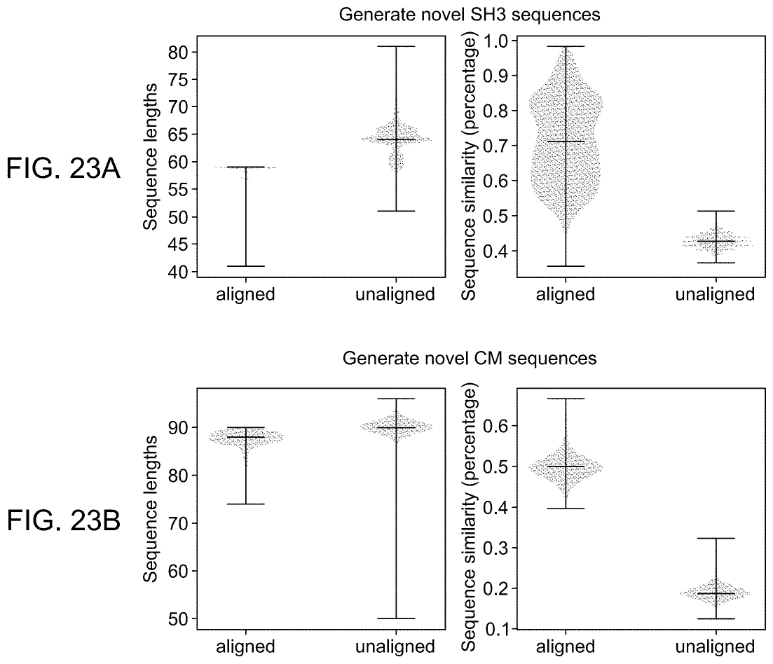

FIGS. 23 and 23B shows an analysis of the entire pool of generated sequences from the SH3 and CM tasks. The sequence length of each sequence is computed for the SH3 task (FIG. 23A), finding that the length variability is more diverse when trained on unaligned versus aligned input data. This was not necessarily the case for the CM homologs (FIG. 23B). However, for both the SH3 and CM tasks, the model is able to generate sequences that are less sequence similar to the natural homologs when trained on unaligned input data (see FIGS. 23A and 23B), illustrating a potential advantage of training generative models on unaligned sequences in generating a broader diversity of sequences that better recapitulate the natural diversity of sequence lengths while maintaining the native tertiary structure.

Regarding FIGS. 23A and 23B, 5611 and 1130 novel SH3 and CM sequences, respectively, are generated by randomly sampling across the latent space. In FIG. 23A, the generated sequences are compared in terms of sequence length and similarity to the nearest natural SH3 homolog for models trained on aligned or unaligned input data. Similarly in FIG. 23B, the generated sequences' lengths and similarity are compared to the nearest natural CM homolog. For both SH3 and CM, sequence diversity is improved when trained on unaligned sequences, but the sequence length variability for generated CM homolog is similar regardless of aligned or unaligned input data.

Results: Fitness Prediction

One important goal of deep learning models for biology is to learn meaningful representations that can be leveraged on down-stream tasks. For instance, a major endeavor in protein design is fitness landscape prediction and representation learning for semi- and self-supervised tasks. The deep generative model is extended to a semi-supervised paradigm in the hopes of learning biologically meaningful representations for fitness landscape prediction. A main goal is to learn a latent space z that is informative for the generative and discriminative tasks. The intuition behind this construction is based on the ideal that the representations that can be used to reconstruct the training data and generate new data indistinguishable from the training data can also be more meaningful for discriminative tasks (e.g. fitness landscape prediction). To benchmark the model's learned representations, the ability to predict test datasets is predicted on four main protein systems from two popular community benchmarks tasks: TAPE and FLIP. See Roshan Rao, Nicholas Bhattacharya, Neil Thomas, Yan Duan, Peter Chen, John Canny, Pieter Abbeel, and Yun Song, “Evaluating protein transfer learning with TAPE,” Advances in neural information processing systems, 32, 2019, incorporated herein by reference in its entirety. Thus, the four protein systems are the following:
1. Mutational screening fitness landscape of VP-I AAV proteins (FLIP). See Drew H Bryant, Ali Bashir, Sam Sinai, Nina K Jain, Pierce J Ogden, Patrick F Riley, George M Church, Lucy J Colwell, and Eric D Kelsic, “Deep diversification of an AAV capsid protein by machine learning,” Nature Biotechnology, 39(6):691-696, 2021; and Ran Zhang, Lin Cao, Mengtian Cui, Zixian Sun, Mingxu Hu, Rouxuan Zhang, William Stuart, Xiaochu Zhao, Zirui Yang, Xueming Li, et al., “Adeno-associated virus 2 bound to its cellular receptor aavr,” Nature microbiology, 4(4):675-682, 2019, each incorporated herein by reference in their entirety.
2. Highly epistatic mutational landscape GB1 (FLIP). See Nicholas C Wu, Lei Dai, C Anders Olson, James O Lloyd-Smith, and Ren Sun, “Adaptation in protein fitness landscapes is facilitated by indirect paths,” Elife, 5:e16965, 2016; and W Trent Franks, Benjamin J Wylie, Sara A Stellfox, and Chad M Rienstra, “Backbone conformational constraints in a microcrystalline u-15n-labeled protein by 3d dipolar-shift solid-state nmr spectroscopy,” Journal of the American Chemical Society, 128(10):3154-3155, 2006, each incorporated herein by reference in their entirety.
3. Epistatic Green Fluorescent Protein (GFP) Landscape Predictions (TAPE). See Karen S Sarkisyan, Dmitry A Bolotin, Margarita V Meer, Dinara R Usmanova, Alexander S Mishin, George V Sharonov, Dmitry N Ivankov, Nina G Bozhanova, Mikhail S Baranov, Onuralp Soylemez, et al., “Local fitness landscape of the green fluorescent protein,” Nature, 533(7603):397-401, 2016, incorporated herein by reference in its entirety.
4. Stability Landscape Prediction (TAPE). See Gabriel J Rocklin, Tamuka M Chidyausiku, Inna Goreshnik, Alex Ford, Scott Houliston, Alexander Lemak, Lauren Carter, Rashmi Ravichandran, Vikram K Mulligan, Aaron Chevalier, et al., “Global analysis of protein folding using massively parallel design, synthesis, and testing,” Science, 357 (6347):168-175, 2017, incorporated herein by reference in its entirety.

Benchmark Model on Landscape Prediction Tasks

For the FLIP AAV capsid task, there were 7 different data split tasks, which are each relevant to protein engineering scenarios. The semi-supervised model is able to outperform or competitively perform to current baseline scores in 5 out of 6 dataset splits (Tables 4 and 5). However, the semi-supervised generative model underperforms when the training set contains only low-fitness sequences and the test set contains only high-fitness sequences. For the FLIP GB1 task, the model outperforms or competitively performs against the current baseline scores (Table 1). However, similar to the AAV capsid tasks, the model underperforms on the protein task where the training and testing splits contain only low- and high-fitness sequences. For the TAPE tasks, the model competitively performs against the state-of-the-art models on the GFP tasks (Table 6) while also performing competitively against the dark and bright GFP modes for the GFP task (Tables 8, 9). However, the model underperforms on the stability prediction task found in Table 7. Overall, these results suggest that the generative model is not only capable of unsupervised learning and protein design, but also capable of generalizing such that the model can infer fitness landscapes and predict function from sequence alone, and is competitive with state-of-the-art methods.

TABLE 4
AAV performance comparison to current baseline scores
(metric: Spearman correlation).

| | Mut-des | des-mut | low-vs-high |
|---|---|---|---|
| Architecture | ρ | ρ | ρ |
| ESM-1b | 0.76 | N/A | 0.39 |
| ESM-1v | 0.79 | N/A | 0.34 |
| Ridge | 0.64 | 0.53 | 0.12 |
| CNN | 0.71 | 0.75 | 0.34 |
| Levenshtein | 0.60 | −0.07 | 0.25 |
| BLOSUM62 | N/A | N/A | N/A |
| The model | 0.82 | 0.78 | 0.17 |

TABLE 5
AAV performance comparison to current baseline scores on
mutagenesis-based dataset splits (metric: Spearman correlation).

| | 1-vs-rest | 2-vs.rest | 7-vs-rest |
|---|---|---|---|
| Architecture | ρ | ρ | ρ |
| ESM-1b | 0.03 | 0.65 | 0.65 |
| ESM-1v | 0.10 | 0.70 | 0.70 |
| Ridge | 0.22 | 0.03 | 0.65 |
| CNN | 0.48 | 0.74 | 0.74 |
| Levenshtein | −0.11 | 0.57 | 0.53 |
| BLOSUM62 | N/A | N/A | N/A |
| The model | 0.61 | 0.74 | 0.71 |

TABLE 6
GFP state-of-the-art scores (metrics: mean squared error MSE and spearman correlation
ρ). Here, the metrics are evaluated on both the bright and dark modes. In Table 8 and 9, the metrics
are evaluated on the bright and dark mode of the test set.

| Architecture | Pretraining | Full MSE | Full ρ |
|---|---|---|---|
| TAPE Transformer | no pretraining | 2.59 | 0.22 |
| TAPE LSTM | no pretraining | 2.35 | 0.21 |
| TAPE ResNet | no pretraining | 2.79 | −0.28 |
| ESM | masked language | N/A | 0.68 |
| TAPE Transformer | masked language | 0.22 | 0.68 |
| TAPE LSTM | bidirectional language | 0.19 | 0.67 |
| TAPE ResNet | masked language | 3.04 | 0.21 |
| UniRep | language + structure | 0.20 | 0.67 |
| LSTM | supervised | 2.17 | 0.33 |
| CPCProt | contrastive | N/A | 0.68 |
| CPCProt-LSTM | contrastive | N/A | 0.68 |
| Linear regression | none | 0.35 | 0.68 |
| CNN | none | 0.23 | 0.68 |
| Mutation count | none | N/A | 0.45 |
| BLOSUM62 score | none | N/A | 0.50 |
| The model | no pretraining | 0.21 | 0.67 |

See Tristan Bepler and Bonnie Berger, “Learning protein sequence embeddings using information from structure,” arXiv preprint arXiv:1902.08661, 2019; Amy X Lu, Haoran Zhang, Marzyeh Ghassemi, and Alan M Moses, “Self-supervised contrastive learning of protein representations by mutual information maximization,” BioRxiv, 2020; Amir Shanehsazzadeh, David Belanger, and David Dohan, “Is transfer learning necessary for protein landscape prediction?” arXiv preprint arXiv:2011.03443, 2020, each incorporated herein by reference in their entirety.

TABLE 7
Overall stability prediction results on the test set
(metrics: Spearman's correlation ρ and accuracy)

| Architecture | Spearman's ρ | Accuracy |
|---|---|---|
| Transformer (No pretraining) | −0.06 | 0.5 |
| LSTM (No pretraining) | 0.28 | 0.6 |
| ResNet (No pretraining) | 0.61 | 0.68 |
| Transformer (Pretrained) | 0.73 | 0.70 |
| LSTM (Pretrained) | 0.69 | 0.69 |
| RexNet (Pretrained) | 0.73 | 0.66 |
| Supervised | 0.64 | 0.67 |
| UniRep | 0.73 | 0.69 |
| Baseline | 0.19 | 0.58 |
| The model | 0.51 | N/A |

TABLE 8
GFP benchmark scores on the bright mode only
(metrics: mean-squared error and Spearman's ρ).

| | | Bright | Bright |
|---|---|---|---|
| Architecture | Pretraining | MSE | ρ |
| TAPE Transformer | no pretraining | 0.08 | 0.08 |
| TAPE LSTM | no pretraining | 0.11 | 0.05 |
| TAPE ResNet | no pretraining | 0.07 | −0.07 |
| ESM | masked language | N/A | N/A |
| TAPE Transformer | masked language | 0.09 | 0.60 |
| TAPE LSTM | bidirectional language | 0.12 | 0.62 |
| TAPE ResNet | masked language | 0.12 | 0.05 |
| UniRep | language + structure | 0.13 | 0.63 |
| LSTM | supervised | 0.08 | 0.06 |
| CPCProt | contrastive | N/A | N/A |
| CPCProt-LSTM | contrastive | N/A | N/A |
| Linear regression | none | 0.09 | 0.68 |
| CNN | none | 0.12 | 0.66 |
| Mutation count | none | N/A | N/A |
| BLOSUM62 scom | none | N/A | N/A |
| The model | no pretraining | 0.118 | 0.54 |

TABLE 9
GFP benchmark scores on the dark mode only
(metrics: mean-squared error and Spearman's g).

| | | Dark | Dark |
|---|---|---|---|
| Architecture | Pretraining | MSE | ρ |
| TAPE Transformer | no pretraining | 3.79 | 0 |
| TAPE LSTM | no pretraining | 3.43 | −0.01 |
| TAPE ResNet | no pretraining | 4.1 | −0.01 |
| ESM | masked language | N/A | N/A |
| TAPE Transformer | masked language | 0.29 | 0.05 |
| TAPE LSTM | bidirectional language | 0.22 | 0.04 |
| TAPE ResNet | masked language | 4.45 | 0.02 |
| UniRep | language + structure | 0.24 | 0.04 |
| LSTM | supervised | 3.17 | 0.02 |
| CPCProt | contrastive | N/A | N/A |
| CPCProt-LSTM | contrastive | N/A | N/A |
| Linear regression | none | 0.33 | 0.05 |
| CNN | none | 0.28 | 0.05 |
| Mulation count | none | N/A | N/A |
| BLOSUM62 score | none | N/A | N/A |
| The model | no pretraining | 0.27 | 0.06 |

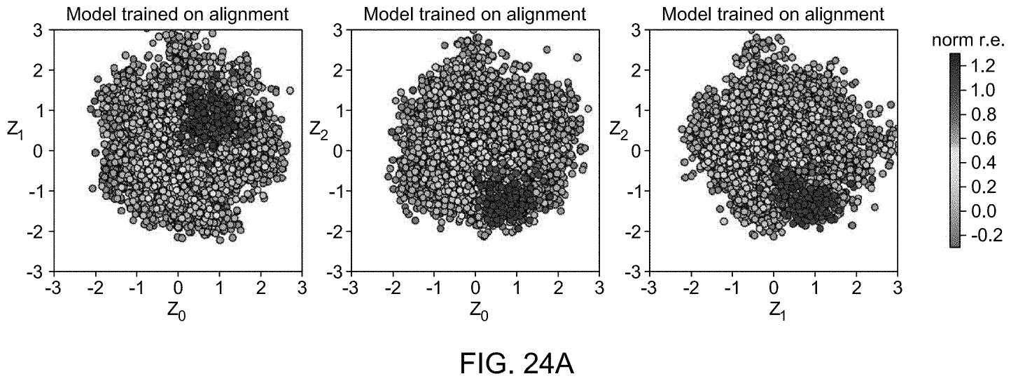

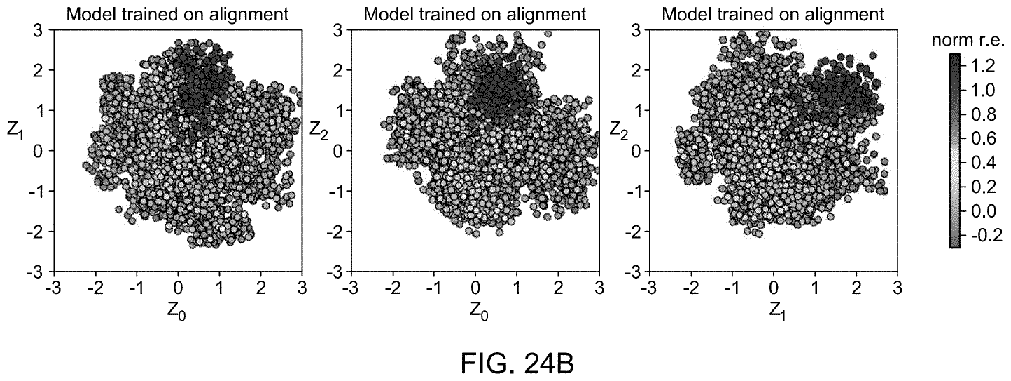

Additional 2D projections of the SH3 dataset is shown on FIGS. 24A and 24B, which show all of the possible 2D projections of the SH3 latent space. FIG. 24A shows the latent embeddings of the natural homologs when the model is trained on aligned input data, while FIG. 24B shows the latent embeddings of natural homologs when the model is trained on unaligned input data. Regardless, whether the model is trained on aligned or unaligned input data, the encoder learns an embedding that discriminates between high activity orthologs (red) from low activity paralogs (blue). Importantly, no functional assay data was provided to the model during training and the unsupervised model learned this partitioning based on sequence data alone.

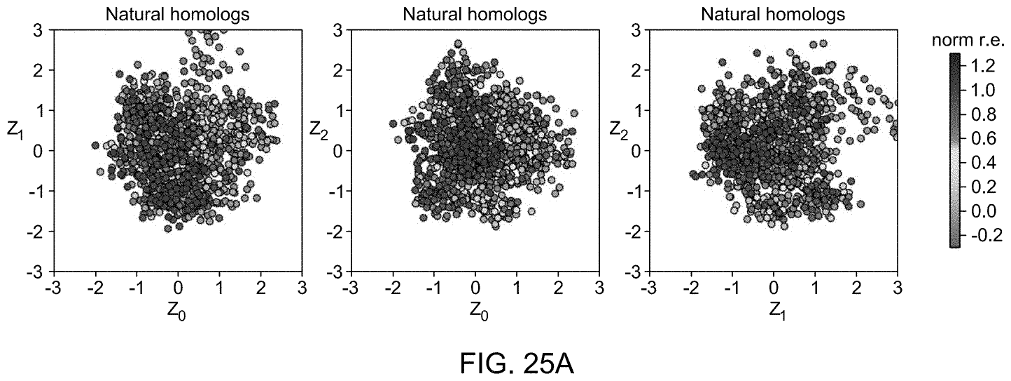

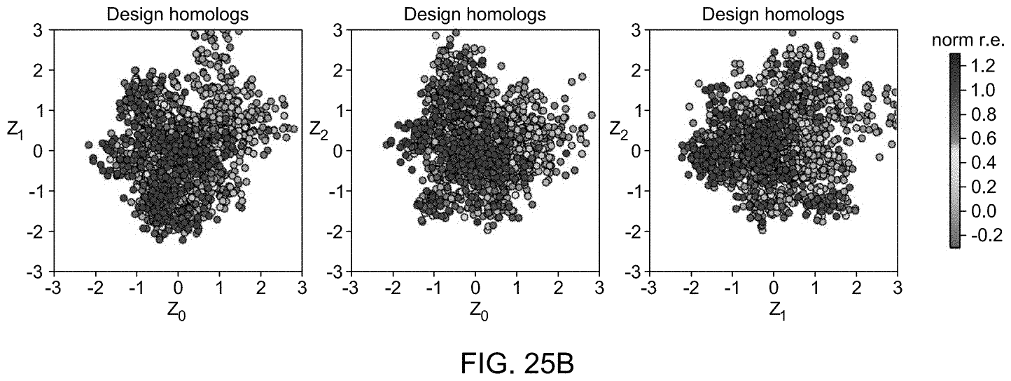

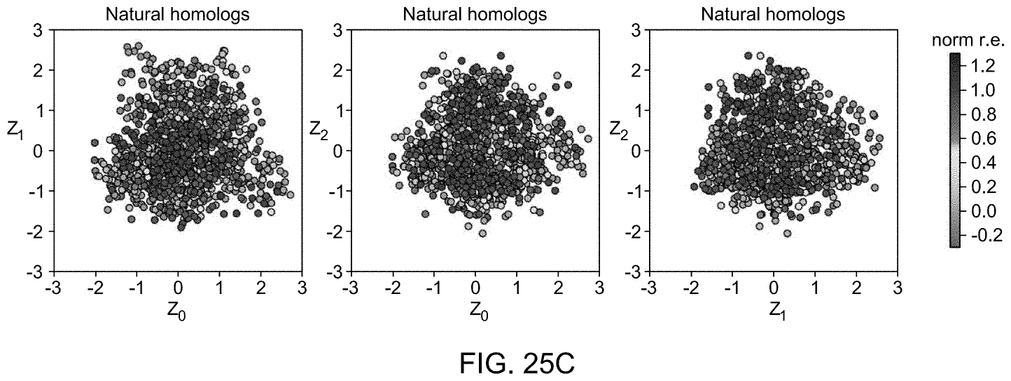

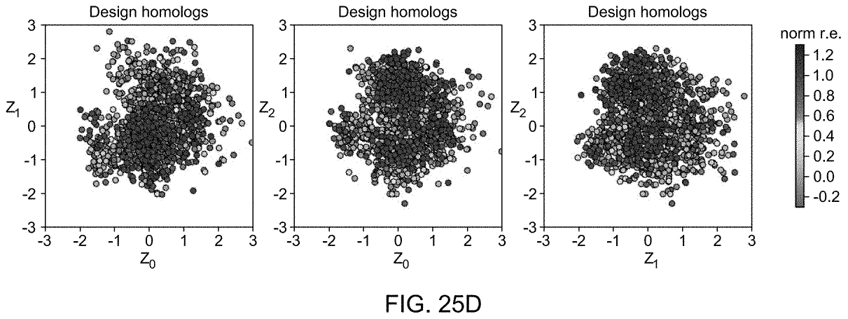

Additional 2D elevations of the CM latent space are provided in FIGS. 25A, 25B, 25C, and 25D, which show all of the possible 2D projections of the CM latent space. FIGS. 25A and 25B show the latent embeddings of the natural and synthetic design homologs when the model is trained on aligned input data, while FIGS. 25C and 25D show the latent embeddings of natural and synthetic design homologs when the model is trained on unaligned input data. Regardless, whether the model is trained on aligned or unaligned input data, the encoder is able to learn a latent space that retains the Gaussian structure and learns representations, clusters of high fitness regions. Importantly, the synthetic design sequences are data from the hold-out set, demonstrating the model's ability to generalize.

Thus, in some of the disclosed embodiments, a variational autoencoder (VAE) and autoregressive generator (WaveNet) are combined for protein design, avoiding the need of multiple-sequence aligned input data. To avoid posterior collapse when combining VAEs and WaveNet models, an Infomation Maximizing VAE (InfoVAE) is implemented, adding a mutual information term to the common ELBO training objective, improving amortized inference, and forcing the decoder to use the latent conditional information. The generative model is able to learn meaningful latent space representations from homologous protein families, which can be leveraged to design novel functional sequences with length variability. The generated structures can be realized by sequence designs that are predicted by AlphaFold2 to adopt tertiary structures in good agreement with the native fold. Additionally, when the model is trained on unaligned versus aligned input sequences, the diversity of the generated sequences can improve, illustrating a potential advantage of using an autoregressive decoder. The model is extended to a semi-supervised learning paradigm and benchmarked the model on four different fitness landscape prediction tasks from FLIP and TAPE. The model can outperform many baseline scores for the AAV and GB1 tasks. In addition, the model can compete against state-of-the-art performance for the GFP task. These results and analysis suggest the deep generative model is capable of successful protein design of variable length sequences, inferring meaningful biological representations, and effectively predicting fitness from sequence alone.

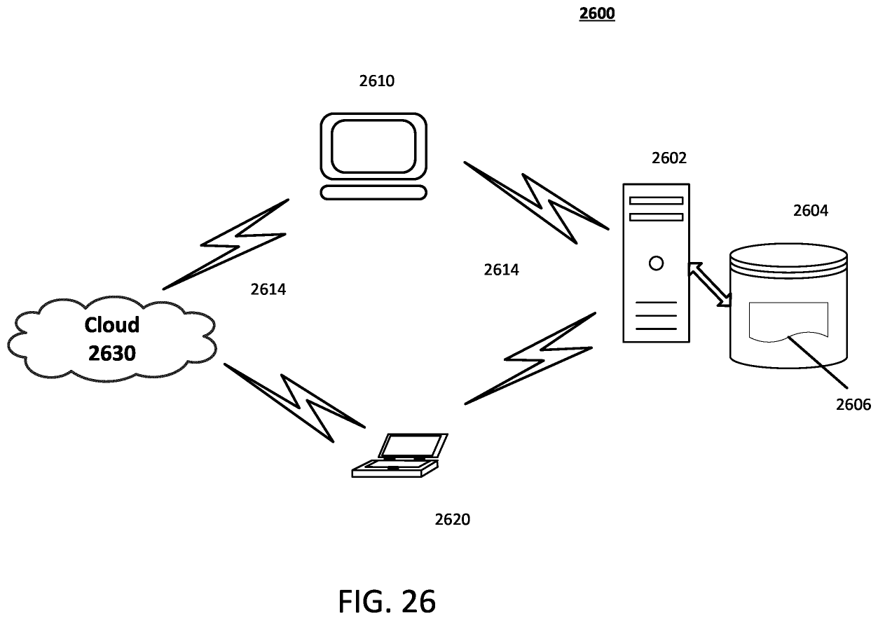

FIG. 26 is a block diagram of a system environment in which the method can be performed. A system environment 2600 as depicted in FIG. 26 can include one or more user computer devices 2610, 2620, where a user can interact with the system 2600. The user computer devices 2610, 2620 may be connected to the system by way of known network connections, such as wireless or wired connections 2614. The system 2600 may include one or more server computers 2602 which may be high-end workstations for performing machine learning algorithms. The one or more server computers 2602 may include, or be connected to, a database system 2604. The database system 2604 may store files 2606 containing data, such as training and testing data used in machine learning. In some embodiments, the system environment 2600 may include connection via the Internet to a cloud service 2630. The cloud service 2630 may be configured to perform machine learning algorithms, as well as provide a database management system service.

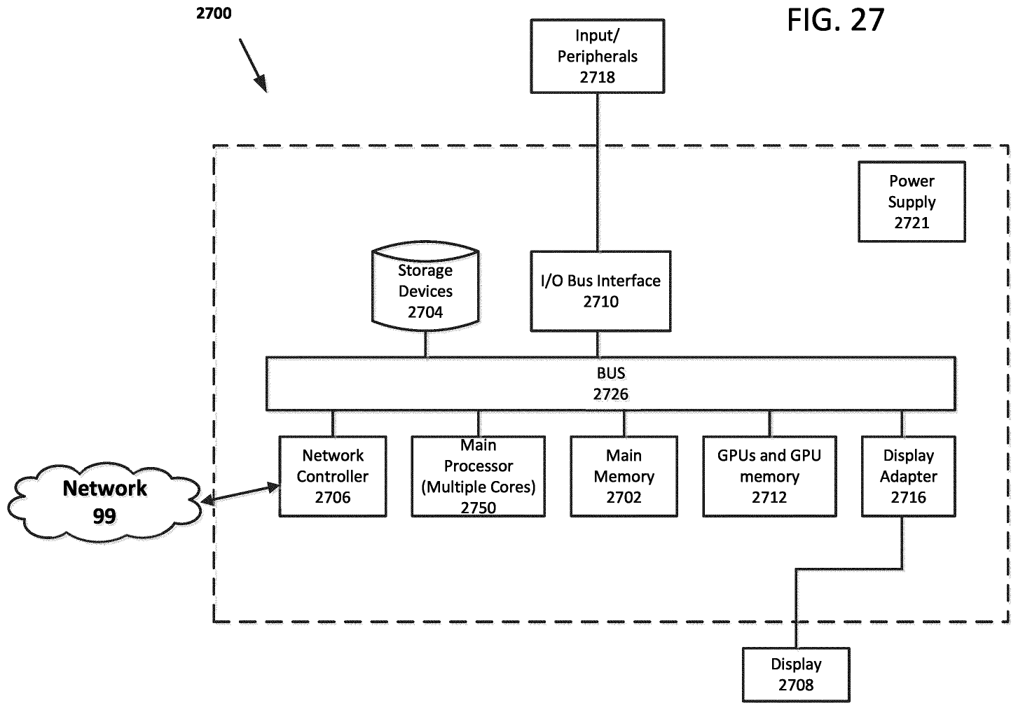

FIG. 27 is a block diagram illustrating an example computer system for implementing the machine learning training and inference methods according to an exemplary aspect of the disclosure. The computer system may be an Al workstation running an operating system, for example Ubuntu Linux OS, Windows Server, a version of Unix OS, or Mac OS Server. The computer system 2700 may include one or more central processing units (CPU) 2750 having multiple cores. The computer system 2700 may include a graphics board 2712 having multiple GPUs, each GPU having GPU memory. The graphics board 2712 may perform many of the mathematical operations of the disclosed machine learning methods. The computer system 2700 includes main memory 2702, typically random access memory RAM, which contains the software being executed by the processing cores 2750 and GPUs 2712, as well as a computer-readable non-volatile storage device 2704 for storing data and the software programs for the above methods. Several interfaces for interacting with the computer system 2700 may be provided, including an I/O Bus Interface 2710, Input/Peripherals 2718 such as a keyboard, touch pad, mouse, Display Adapter 2716 and one or more Displays 2708, and a Network Controller 2706 to enable wired or wireless communication through a network 99. The interfaces, memory and processors may communicate over the system bus 2726. The computer system 2700 includes a power supply 2721, which may be a redundant power supply.

In some embodiments, the computer system 2700 may include a server CPU and a graphics card by NVIDIA, in which the GPUs have multiple CUDA cores.

The above disclosure also encompasses the embodiments listed below.

(1) A method of generating protein sequences. The method includes
encoding, using a dilated convolutional encoder, a plurality of input protein sequences onto a latent space distribution; and
decoding, using a decoder employing dilated causal convolutions, the latent space distribution to generate new protein sequences different from the input protein sequences.

(2) A method of generating protein sequences using a system including an encoder coupled to an autoregressive generator, and having been trained with a loss function that comprises reconstruction loss and a mutual information maximization term. The method includes
encoding, using the encoder, a plurality of input protein sequences onto a latent space distribution; and
decoding, using the autoregressive generator, the latent space distribution to generate new protein sequences different from the input protein sequences.

(3) The method of feature (2), in which the decoding step comprises decoding the latent space distribution using a dilated casual convolution autoregressive generator as the autoregressive generator.

(4) The method of feature (2), in which the decoding step comprises decoding the latent space distribution to generate the new protein sequences, which include sequences of different lengths.

(5) The method of feature (2), in which the encoding step comprises encoding the plurality of input protein sequences, which are unaligned.

(6) The method of feature (2), in which the encoding step comprises encoding the plurality of input protein sequences using a dilated convolutional neural network encoder.

(7) The method of feature (2), in which the encoding step comprises encoding the plurality of input protein sequences into a latent space embedding.

(8) The method of feature (2), in which the decoding step comprises predicting a next amino acid in a particular sequence, based on the particular sequence and a latent space embedding.

(9) The method of feature (2), in which the system was trained using the loss function, which further includes a semi-supervised loss.

(10) The method of any of features (2) or (3), in which the decoding step further comprises decoding the latent space distribution using the dilated casual convolution autoregressive generator, which incorporates residual and skip connections.

(11) A method of training a system for generating protein sequences, the system including an encoder that encodes a plurality of input protein sequences onto a latent space distribution, and an autoregressive generator that decodes the latent space distribution to generate new protein sequences different from the input protein sequences. The method includes training the system with a loss function that comprises reconstruction loss and a mutual information maximization term.

(12) The method of feature (11), in which the system further includes a semi-supervised learning module including a regression model with a set of training parameters that are learned by minimizing, for a subset of the latent space distribution, an error between outputs of the regression model and fitness values obtained from assay measurements; and the method further includes training the system with a modified loss function that further includes term based on performance of the regression model.

(13) The method of feature (12), in which the term in the modified loss function is a mean-squared error term based on a ground truth and a predicted regression value of the regression model.

(14) The method of feature (12), in which the regression model is a neural network having weights as the training parameters, which are determined in the training step.

(15) The method of feature (11), in which the encoder is a dilated convolutional neural network encoder.

(16) The method of feature (11), in which the encoder learns a latent space embedding.

(17) The method of feature (11), in which the training step comprises training the system with the loss function, which further includes a semi-supervised loss.

(18) A system for generating protein sequences, including
an encoder configured to encode a plurality of input protein sequences onto a latent space distribution; and
an autoregressive generator configured to decode the latent space distribution to generate new protein sequences different from the input protein sequences,
in which the system is trained with a loss function that includes reconstruction loss and a mutual information maximization term.

(19) The system of feature (18), in which the autoregressive generator is a dilated casual convolution autoregressive generator.

(20) The system of feature (18), in which the autoregressive generator is further configured to decode the latent space distribution to generate the new protein sequences, which include sequences of different lengths.

(21) The system of feature (18), in which the encoder is further configured to encode the plurality of input protein sequences, which are unaligned.

(22) The system of feature (18), in which the system was trained with the loss function, which further includes a semi-supervised loss.

(23) The system of feature (19), in which the dilated casual convolution autoregressive generator incorporates residual and skip connections.

(24) A non-transitory computer-readable medium storing a program that, when executed by processing circuitry, causes the processing circuitry to perform a method of generating protein sequences using a system including an encoder coupled to an autoregressive generator, and having been trained with a loss function that includes reconstruction loss and a mutual information maximization term. The method includes
encoding, using the encoder of a variational autoencoder, a plurality of input protein sequences onto a latent space distribution; and
decoding, using the autoregressive generator, the latent space distribution to generate new protein sequences different from the input protein sequences.

1. A method of generating protein sequences, the method comprising:
encoding, using a dilated convolutional encoder, a plurality of input protein sequences onto a latent space distribution; and
decoding, using a decoder employing dilated causal convolutions, the latent space distribution to generate new protein sequences different from the input protein sequences.

2. A method of generating protein sequences using a system including an encoder coupled to an autoregressive generator, and having been trained with a loss function that comprises reconstruction loss and a mutual information maximization term, the method comprising:
encoding, using the encoder, a plurality of input protein sequences onto a latent space distribution; and
decoding, using the autoregressive generator, the latent space distribution to generate new protein sequences different from the input protein sequences.

3. The method of claim 2, wherein the decoding step comprises decoding the latent space distribution using a dilated casual convolution autoregressive generator as the autoregressive generator.

4. The method of claim 2, wherein the decoding step comprises decoding the latent space distribution to generate the new protein sequences, which include sequences of different lengths.

5. The method of claim 2, wherein the encoding step comprises encoding the plurality of input protein sequences, which are unaligned.

6. The method of claim 2, wherein the encoding step comprises encoding the plurality of input protein sequences using a dilated convolutional neural network encoder.

7. The method of claim 2, wherein the encoding step comprises encoding the plurality of input protein sequences into a latent space embedding.

8. The method of claim 2, wherein the decoding step comprises predicting a next amino acid in a particular sequence, based on the particular sequence and a latent space embedding.

9. The method of claim 2, wherein the system was trained using the loss function, which further includes a semi-supervised loss.

10. The method of claim 3, wherein the decoding step further comprises decoding the latent space distribution using the dilated casual convolution autoregressive generator, which incorporates residual and skip connections.

11. A method of training a system for generating protein sequences, the system including an encoder that encodes a plurality of input protein sequences onto a latent space distribution, and an autoregressive generator that decodes the latent space distribution to generate new protein sequences different from the input protein sequences, the method comprising:
training the system with a loss function that comprises reconstruction loss and a mutual information maximization term.

12. The method of claim 1, wherein the system further includes a semi-supervised learning module including a regression model with a set of training parameters that are learned by minimizing, for a subset of the latent space distribution, an error between outputs of the regression model and fitness values obtained from assay measurements; and
the method further comprises training the system with a modified loss function that further includes term based on performance of the regression model.

13. The method of claim 12, wherein the term in the modified loss function is a mean-squared error term based on a ground truth and a predicted regression value of the regression model.

14. The method of claim 12, wherein the regression model is a neural network having weights as the training parameters, which are determined in the training step.

15. The method of claim 11, wherein the encoder is a dilated convolutional neural network encoder.

16. The method of claim 11, wherein the encoder learns a latent space embedding.

17. The method of claim 11, wherein the training step comprises training the system with the loss function, which further includes a semi-supervised loss.

18. A system for generating protein sequences, comprising:
an encoder configured to encode a plurality of input protein sequences onto a latent space distribution; and
an autoregressive generator configured to decode the latent space distribution to generate new protein sequences different from the input protein sequences,
wherein the system is trained with a loss function that includes reconstruction loss and a mutual information maximization term.

19. The system of claim 18, wherein the autoregressive generator is a dilated casual convolution autoregressive generator.

20. The system of claim 18, wherein the autoregressive generator is further configured to decode the latent space distribution to generate the new protein sequences, which include sequences of different lengths.

21. The system of claim 18, wherein the encoder is further configured to encode the plurality of input protein sequences, which are unaligned.

22. The system of claim 18, wherein the system was trained with the loss function, which further includes a semi-supervised loss.

23. The system of claim 19, wherein the dilated casual convolution autoregressive generator incorporates residual and skip connections.

24. A non-transitory computer-readable medium storing a program that, when executed by processing circuitry, causes the processing circuitry to perform a method of generating protein sequences using a system including an encoder coupled to an autoregressive generator, and having been trained with a loss function that includes reconstruction loss and a mutual information maximization term, the method comprising:
encoding, using the encoder of a variational autoencoder, a plurality of input protein sequences onto a latent space distribution; and
decoding, using the autoregressive generator, the latent space distribution to generate new protein sequences different from the input protein sequences.
